(19) United States
(12) Patent Application Publication
(10) Pub. No.: US 2006/0057154 A1
(43) Pub. Date: Mar. 16, 2006

(54) Compositions for inducing of immunotolerance

(75) Inventors: Antonius Josephus Maria van Oosterhout, Groningen (NL); Martien Lukas Kapsenberg, Amsterdam (NL); Frank Reinoud Weller, Abcoude (NL); Yousef Al-Madane Taher, Zeist (NL); Elisabeth Catharina Adriana Maria Lobato-van Esch, Utrecht (NL); Joost Lambert Max Vissers, Nijmegen (NL)

Correspondence Address: TRASK BRITT, P.O. BOX 2550, SALT LAKE CITY, UT, 84110, US

(73) Assignee: Universiteit Utrecht Holding B.V., Utrech (NL)

(21) Appl. No.: 11/229,333

(22) Filed: Sep. 15, 2005

Related U.S. Application Data
(63) Continuation of application No. PCT/NL04/00205, filed on Mar. 25, 2004.

(30) Foreign Application Priority Data
Mar. 28, 2003 (EP) 03075909.6

(51) Int. Cl. A61K 48/00; A61K 39/00; A61K 31/59; A61K 31/60; A61K 31/573

(52) U.S. Cl. 424/185.1; 514/44; 514/171; 514/167; 514/165; 514/570

(57) ABSTRACT

Described are methods of treating allergic disorders and compositions for use therein. The methods comprise administering an allergen and one or more medicaments. These medicaments are compounds that inhibit the transcription of genes involved in the initiation of innate and specific immunity, thereby promoting the development of tolerance to these allergens, through inhibition of the NF-κB and/or the MAPK/AP-1 signal transduction pathway(s). In another embodiment, the use of DNA vaccines is disclosed that incorporates a gene encoding one or more allergen sequences or fragments thereof, in combination with genes encoding proteins that inhibit the activation of the NF-κB and/or the MAPK/AP-1 pathway or in combination with small interfering RNA sequences or anti-sense sequences that inhibit the expression of NP-κB and/or AP-1 proteins.

### CROSS-REFERENCE TO RELATED APPLICATIONS

This application is a continuation of PCT International Patent Application No. PCT/NL2004/000205, filed on Mar. 25, 2004, designating the United States of America, and published, in English, as PCT International Publication No. WO 2004/084927 A2 on Oct. 7, 2004, which application claims priority to European Patent Application No. 03075909.6 filed on Mar. 28, 2003, the contents of each of which are incorporated herein by this reference.

### TECHNICAL FIELD

The invention relates to the field of immunology, more particularly to the field of immune therapy, such as the induction of tolerance against an allergen, more specifically to the immunization with allergen and inhibiting the production of co-stimulator molecules in antigen-presenting cells. The invention provides methods of treating allergic disorders and compositions for use therein.

### BACKGROUND

Adaptive Immunity and Peripheral Tolerance

Adaptive immunity is initiated by the antigen-specific stimulation of naive T-cells by peptide MHC class I or II complexes expressed by antigen-presenting cells (“APCs,” i.e., dendritic cells, macrophages, monocytes, B-lymphocytes). Effective responses, however, require the additional stimulation of naïve T-cells by “co-stimulator” molecules expressed by these APCs (Baxter et al., 2002; Matzinger, 2002). The expression of “co-stimulator” molecules is part of the innate immune response induced by biologically active environmental substances (such as a pathogen or any biologically active compound, including an allergen) that affect migratory non-specific immune cells and/or resident tissue cells. The expression in APCs is induced directly by the biological activity of environmental substances and/or indirectly by the reactivity products (stress and inflammatory mediators, necrotic cells) generated in response to these substances by other cells within the APC tissue micro-environment (Gallucci et al., 2001).

The reactivity of non-specific immune cells, such as APCs, and resident cells to these biologically active compounds, is part of the innate immune response and triggered in most, if not all, cases via the NF-κB and/or the MAPK/AP-1 signal transduction pathways. Innate immunity activation pathways are triggered by compounds including but not limited to:
1. “Pathogen-associated molecular patterns” (PAMPs) present in endotoxins, peptidoglycans, carbohydrates and other microbial constituents by so-called “pattern recognition receptors” (PRR) including the toll-like receptors, scavenger receptors and lectin receptors (Pulendran et al., 2001; Reis e Sousa, 2001). PRR recognize not only exogenous molecules but also endogenous molecules such as heat-shock proteins and hyaluronan.
2. Mediators of oxidative stress generated within the APC microenvironment.
3. Heat-shock proteins affecting APCs through interaction with different cell-membrane receptors such as CD91 and toll-like receptors -2 and -4 (Gallucci et al., 2001).
4. Intracellular nucleotides like ATP and UTP (Gallucci et al., 2001).
5. Proteolytic enzymes that are locally released from resident cells or provided by the antigen itself and may activate these transcription factors through the protease-activated receptor family. In this respect, it is important to note that many allergens have proteolytic activities (Gallucci et al., 2001). Moreover, allergens may also directly activate NF-κB in airway epithelial cells and allergen challenge rapidly activates NF-κB in airway epithelium in an animal model of asthma.
6. The family of cytosolic pattern recognition receptors for intracellular pathogens called nucleotide-binding oligomerization domain (NOD), also called caspase recruitment domain (CARD).

The NF-κB family of transcription factors and the transcription factor AP-1 have a central role in coordinating the expression of a wide variety of genes that control immune and inflammatory responses, including cytokines, chemokines, cell-adhesion molecules, co-stimulatory molecules, complement factors and anti-apoptotic factors (Herlaar et al., 1999; McKay et al., 1999). Whereas these molecules are central in the innate immune responses, they also initiate and tailor adaptive immune responses to these compounds by stimulating APC, in particular, dendritic cells (DC), to express “co-stimulation” molecules that effectively alarm naïve T-cells. DC reside in peripheral tissues as highly endocytic immature cells with low expression of co-stimulatory molecules. Activation of immature DC by exposure to certain environmental or stress compounds induces their maturation into mature DC with high cell surface expression of MHC molecules complexed with peptides from proteins that have been internalized in their immature stage and high cell surface expression of “co-stimulation” molecules. Therefore, mature DC efficiently activate naïve T-cells specific against peptide-derived proteins from the environmental compounds.

Mature DC promote the development of subsets of immunogenic (such as Th1 or Th2) and/or tolerogenic (such as the regulatory cells Treg, Tr1 or Th3) T-cells, but the balance of these subsets strongly depends on the way the mature DC have been activated in their immature stage. Since different pathogens activate immature DC in different ways resulting in different functional phenotypes of mature DC, DC can tailor the class of specific immune response to the invading pathogen. Ideally, this process results in protection against this pathogen by immunogenic T-cells without lethal pathology to host tissue induced by the activity of these Th-cells. The selective development of T-cell subsets is directed by the selective expression levels of cell-surface molecules such as co-stimulatory molecules (CD40, B7-family proteins and others) and by the production of T-cell skewing cytokines (IL-12, type 1 IFNs, IL-10, TGF-β and others).

Most importantly, in steady-state conditions, in the absence of environmental biologically active compounds or stress reactions, T-cells continuously engage immature DC with low expression “co-stimulation” molecules. Activation of Th-cells in the absence of “co-stimulation” also results in the development of regulatory T-cells that mediate tolerance to auto-antigens ubiquitously carried by these DC, another level of protection against autoimmunity.

Peripheral tolerance can be defined as the failure to respond to an antigen by an adaptive immune response and is acquired by mature lymphocytes in peripheral tissues. Although still incompletely understood, the mechanisms of action of peripheral tolerance can be due to (i) anergy, (ii) immune deviation, (iii) activation-induced cell death (apoptosis) or other at present unknown mechanisms. Recently, regulatory T-lymphocytes have been shown to mediate peripheral tolerance in at least some immunological disease models such as colitis, transplant rejection, allergic- and auto-immune diseases. The family of regulatory T-cells is diverse. They are all anergic, i.e., do not proliferate in response to antigen, and are tolerogenic, i.e., suppress the activity of immunogenic T-cells. In non-pathogenic conditions, Treg are thymus-derived, they suppress immunogenic Th-cells via a cell-contact-dependent mechanism and are important in prevention of auto-immunity. Tr1-cells are characterized by the production of IL-10 and have been shown to suppress both Th1 and Th2 responses and thereby prevents the development of auto-immunity and allergic diseases. Th3-cells are characterized by the production of TGFβ and have been shown to be involved in oral tolerance.

The mechanism by which APCs, in particular DCs, induce the development of regulatory T-cells is largely unknown. Although DC are involved in the generation of these forms of peripheral tolerance, it is at present unknown whether they are a different subset or are generated out of immature DC by (unknown) micro-environmental factors. DC that generate Tr1-cells have been characterized by the production of IL-10, whereas DC that generate Th3-cells have been characterized by the production of TGFβ. Antigen-specific regulatory T-cells producing IL-10 and/or anergic T-cells can also be generated by repetitive stimulation with immature DC that present antigen (Dhodapkar et al., 2001; Jonuleit et al., 2001; Roncarolo et al., 2001). In vitro experiments suggest that suppression by the regulatory T-cells induced by partially matured DC is cell-contact dependent (Jonuleit et al., 2001).

Allergen, Vaccination

Although allergen vaccination has been practiced since 1911, recent developments in purification of extracts and in understanding of the mechanism have increased its applicability at present and its promise for the future in the treatment of allergic diseases. Subcutaneous injection with increasing doses of allergen leads in the majority of allergic patients to reduction of allergen-induced inflammation, in a significant reduction in allergic symptoms and medication requirement and improvement in lung function as has been summarized in a meta-analysis. The clinical and anti-inflammatory (skin, conjunctivae) effects lasts even years after stopping the vaccination schedule.

Although this classical form of allergen vaccination is clearly beneficial for the treatment of mono-allergic asthma patients, it seldom results in complete alleviation of all symptoms. Moreover, occurrence of side effects at high doses, especially in asthma, the cumbersome application by repetitious injections during long periods of time, and the strong clinical effects of concurrent therapies such as inhaled corticosteroids, discourage physicians to use it at a large scale. Thus, there is strong need for novel strategies that improve allergen vaccination and reduce unwanted side effects.

Mechanisms of Allergen Vaccination in Allergic Patients

The clinical effect of allergen vaccination (also called allergen-specific immunotherapy or immunotherapy) is likely to be mediated through reduction of allergen-induced inflammation. The immunological process underlying this effect remains at present unknown. As T-cells regulate the inflammatory response, much effort has been focused on activation and differentiation of T-lymphocytes in relation to allergen vaccination. Both allergen-specific hypo-reactivity, as a shift in the cytokine profile of the reacting T-lymphocytes (Th2 to Th1) have been proposed. This has opened the possibility to improve the efficacy of allergen vaccination by immunoregulatory cytokines such as IL-12 or IL-18. Recently, a potential role of IL-10 in the beneficial effect of allergen vaccination in bee-venom allergic patients has been demonstrated. During bee-venom immunotherapy, the reduction of T-cell proliferation and cytokine responses (IL-5, IL-13) upon restimulation in vitro could be fully antagonized by neutralization of IL-10. These data suggest a role for IL-10 producing regulatory T-cells (Tr1) in allergen vaccination. IL-10 has been shown to decrease IgE production and to enhance IgG4 production in human B-lymphocytes in vitro. At the T-cell level, IL-10 reduces T-cell responses to specific antigens by suppressing co-stimulatory signals (B7-1 and B7-2) delivered by antigen-presenting cells.

Pre-Clinical Model of Allergic Asthma in the Mouse

Previously, we have developed a highly reproducible model in the mouse (BALB/c) with immunological and pathophysiological features reminiscent of allergic asthma, e.g., antigen-specific IgE, eosinophilic airway inflammation and airway hyper-responsiveness to methacholine. These asthma features are associated with the appearance of Th2-cells in lung tissue and the draining lymph nodes. The Th2-cells, and the cytokines they produce, play a central role in the initiation and progression of the airway manifestations of asthma as shown by studies using monoclonal antibodies to cytokines and by depletion or transfer of T-cell subsets.

### DISCLOSURE OF THE INVENTION

Allergen Vaccination in a Mouse Model

We were the first to demonstrate that allergen vaccination is effective in a mouse model of allergic asthma. A protocol of subcutaneous allergen injections resembling a semi-rush protocol used in humans was effective to prevent allergen-induced airway hyperreactivity to methacholine and eosinophilic airway inflammation. During allergen vaccination, an initial rise in serum IgE levels occurred, after which IgE levels decreased sharply concomitant with an increase in IgG2a levels. The increase in IgG2a antibodies indicates a role for IFNγ produced by either Th1-cells or Tr1-cells. The down-regulation of these airway manifestations of asthma was associated with decreased Th2 type cytokine production (IL-4 and IL-5) upon in vitro restimulation. These data suggest that the beneficial effect of allergen vaccination is mediated by an effect on Th2-lymphocytes such as (i) anergy, (ii) induction of Th1 or Th3/Tr1-cells (“immune deviation”), (iii) activation-induced cell death (apoptosis) or other at present unknown mechanisms.

In certain embodiments, the present invention provides methods of treating allergic disorders and compositions for use therein. The methods generally comprise administering one or more medicaments with or without administering an allergen. The medicaments decrease the activity of APCs in such a way that the APC is still handling the antigen and exposes the epitopes on its surface to the lymphocyte, but the production of co-stimulator molecules is decreased or prevented. Therefore, in certain embodiments, the invention includes a method to induce and/or increase tolerance to an allergen in a subject, comprising inhibiting and/or preventing the production of a co-stimulator molecule in an antigen-presenting cell in the presence of an allergen.

The allergen may be present in the body already, as is the case with some allergic diseases. In that case, inhibiting or preventing the production of a co-stimulator factor by the antigen-presenting cells alone will enhance the induction of tolerance.

In another embodiment, the allergen is administered to a person in need of such tolerance induction or enhancement. Because the co-stimulator molecules are expressed after triggering of the NF-κB and/or the MAPK/AP-1 signal-transducing pathways, it is an object of the present invention to inhibit the pathways in APCs. Therefore, the present invention teaches a method to induce and/or increase tolerance to an allergen in a subject, comprising inhibiting and/or preventing the production of a co-stimulator molecule in an antigen-presenting cell wherein the production of a co-stimulator molecule is inhibited and/or prevented by inhibiting the NF-κB and/or the MAPK/AP-1 signal-transducing pathways in the antigen-presenting cell, or by inhibiting transcription of genes involved in the activation of the NF-κB and/or the MAPK/AP-1 signal-transducing pathways in an antigen-presenting cell. The invention teaches in Table 1 a number of known compounds that may be put to this new use for this new purpose. Therefore, the invention teaches the method, wherein NF-κB and/or the MAPK/AP-1 signal-transducing pathways in an antigen-presenting cell are inhibited by a ligand to a peroxisome proliferator-activated receptor and/or a functional analogue thereof.

In another embodiment, taught is a method, wherein the NF-κB-transducing pathway is inhibited by at least one anti-oxidant compound and/or proteasome and/or protease inhibitor, IκB phosphorylation and/or degradation inhibitor, and/or a functional analogue thereof.

In yet another embodiment, taught is a method, wherein the NF-κB-transducing pathway is inhibited by at least one non-steroidal anti-inflammatory compound and/or a functional analogue thereof or by at least one glucocorticosteroid compound or by at least one di-hydroxyvitamin D3 compound and/or a functional analogue thereof.

Also disclosed are compounds that can inhibit the MAPK/AP-1 signal-transducing pathway. Therefore, disclosed is a method to induce and/or increase tolerance to an allergen in a subject, the method comprising inhibiting and/or preventing the production of a co-stimulator molecule in an antigen-presenting cell, wherein the production of a co-stimulator molecule is inhibited and/or prevented by inhibiting the NF-κB and/or the MAPK/AP-1 signal-transducing pathways in the antigen-presenting cell, wherein the MAPK/AP-1 signal-transducing pathway is inhibited by at least one non-steroidal and/or steroidal anti-inflammatory compound and/or a functional analogue thereof, or by at least one pyridinylimidazole compound and/or a functional analogue thereof, or by at least one cAMP-elevating compound and/or a functional analogue thereof, or by at least one NF-κB and/or AP-1 decoy oligonucleotide and/or a functional analogue thereof.

The above-mentioned compounds inhibit the transcription of genes involved in the initiation of innate and specific immunity, thereby promoting the development of tolerance to these allergens, through inhibition of the NF-κB and/or the MAPK/AP-1 signal transduction pathway(s). The inhibitor of the MAPK/AP-1 signal-transducing pathways may be given orally, by inhalation or parenteral, or via the skin or a mucosal surface with the purpose of preventing the APCs from producing co-stimulator molecules, thereby inducing tolerance against an allergen.

The inhibitors may be incorporated in a pharmaceutical composition with a suitable diluent. The diluent may be any fluid acceptable for intravenous or parenteral inoculation. In one embodiment, the suitable diluent may comprise water and/or oil and/or a fatty substance. In a preferred embodiment, the inhibitors of the NF-κB pathway are combined with inhibitors of the MAPK/AP-1 pathway. In a more preferred embodiment, the inhibitors are together or by themselves further combined with one or more allergens. Therefore, the present invention teaches a pharmaceutical composition comprising an inhibitor of the NF-κB and/or the MAPK/AP-1 signal-transducing pathway and one or more allergens, further comprising a suitable diluent. The inhibitors may be administrated to a patient in need of such treatment before the administration of the allergens. The inhibitors may be administered via another route than the allergens. For example, the inhibitors may be provided orally, or topically, followed by topical administration of the allergens. Topical administration comprises administration on the skin, and/or on the mucosa of the airways, and/or of the oro-nasal cavity, and/or of the gastro-intestinal mucosa.

In another embodiment, the inhibitors may be combined with allergens before administration to a patient. Therefore, in certain embodiments, the invention also includes a pharmaceutical composition as previously identified herein, wherein the inhibitor of the NF-κB and/or the MAPK/AP-1 signal-transducing pathway is combined with the allergen before administration to a patient. Administration of aforementioned pharmaceutical compositions increases the induction of tolerance to allergens and may diminish disease symptoms in patients suffering from hypersensitivity to various allergens. Therefore, in certain embodiments, the present invention provides a method to increase induction of immunotolerance, comprising providing a pharmaceutical composition as mentioned above by oral, and/or enteral, and/or intranasal, and/or dermal administration.

In another embodiment, the present invention discloses a method to increase induction of immunotolerance, comprising providing an inhibitor of the NF-κB and/or the MAPK/AP-1 signal-transducing pathway by oral, and/or enteral, and/or intranasal, and/or dermal administration, further administering an allergen.

Another approach for inducing tolerance to an allergen is by administering to a patient suffering from hypersensitivity, a DNA sequence that, upon entering a body cell, preferably a cell in the mucosa or dermis, is expressed and a protein or peptide encoded by the DNA fragment is produced. In a more preferred embodiment, the DNA sequence also encodes at least one T-cell epitope, because the presence of such an epitope at the presentation of the allergen (a protein or peptide) to a T-cell enhances the recognition by the T-cell. Therefore, in another embodiment, the present invention discloses a DNA vaccine that incorporates a gene encoding one or more allergen sequences or fragments thereof. For optimal results, the APC should be prevented from producing co-stimulator molecules at or around the time of administration of the above-mentioned DNA vaccine. Therefore, the present invention discloses a method for treating an allergic disease comprising administering a DNA vaccine as mentioned above, further comprising inhibiting the production of a co-stimulator molecule in an antigen-presenting cell. The inhibition of the production of a co-stimulator molecule in an antigen-presenting cell may also be caused by the action of a DNA sequence encoding for a protein that inhibits or prevents the activation of the NF-κB and/or the MAPK/AP-1 signal-transducing pathway. Therefore, the present invention also provides a DNA vaccine comprising a gene encoding one or more allergen sequences, further comprising at least one gene encoding a protein that inhibits the activation of the NF-κB and/or the MAPK/AP-1 signal-transducing pathway.

In yet another embodiment, the inhibition of the production of a co-stimulator molecule in an antigen-presenting cell may be caused by the action of at least one small interfering RNA sequence and/or antisense sequence that inhibits the expression of the NF-κB and/or AP-1 proteins. Therefore, the present invention also provides a DNA vaccine comprising a gene encoding one or more allergen sequences, further comprising at least one small interfering RNA sequence and/or antisense sequence that inhibits the expression of the NF-κB and/or AP-1 proteins. The DNA vaccines can be used to treat patients suffering of allergic disease. Therefore, the present application provides a DNA vaccine as mentioned above for the treatment of allergic disease. Compared to conventional allergen vaccination, these combination methods offer significant advantages, such as (i) better efficacy leading to stronger reduction of symptoms, (ii) reduction of the need for drugs, in particular glucocorticoids, (iii) prevention of the progression into more severe disease, (iv) faster onset of beneficial effects leading to shorter treatment period, (v) use of lower amounts of allergen, and (vi) less unwanted side effects.

### DESCRIPTION OF FIGURES

FIG. 1: Airway responsiveness to inhalation of different doses of methacholine was measured before (A) and after (B) OVA inhalation challenge. Ovalbumin-sensitized BALB/c mice (n=6 per group) were treated with sham-immunotherapy (sham-IT) or OVA-IT alone or in combination with 0.1 μg, 0.03 μg or 0.01 μg 1α, 25(OH)2 VitD3 (VitD3) prior to repeated OVA inhalation challenges. **: P<0.05 as compared to after OVA inhalation challenge; *: P<0.05 as compared to sham-treated mice; #: P<0.05 as compared to OVA-IT alone.

FIG. 2: Serum levels of OVA-specific IgE before (open bars) and after (filled bars) repeated OVA inhalation challenges. *: P<0.05 as compared to before OVA inhalation challenges; #: P<0.05 as compared to sham-IT treated mice; $: P<0.05 as compared to OVA-IT alone.

FIG. 3: Number of eosinophils in bronchoalveolar lavage fluid. #: P<0.05 as compared to sham-IT treated mice; *: P<0.05 as compared to OVA-IT alone.

FIG. 4: Number of eosinophils in bronchoalveolar lavage fluid. #: P<0.05 as compared to sham-IT treated mice; *: P<0.05 as compared to OVA-IT alone.

### DETAILED DESCRIPTION OF THE INVENTION

Inhibition of NF-κB Signal Transduction Pathway

The NF-κB family of transcription factors has a central role in coordinating the expression of a wide variety of genes that control immune and inflammatory responses, including cytokines, chemokines, cell-adhesion molecules, co-stimulatory molecules, complement factors and anti-apoptotic factors (McKay et al., 1999). Mammalian NF-κB family members include RelA (p65), NF-κB1 (p50; p150), NF-κB2 (p52; p100), cRel and RelB. Importantly, experiments with gene-deleted mice have proved that NF-κB1 p50, RelA and cRel are essential in the innate immune function of DC. NF-κB proteins are present in the cytoplasm in association with inhibitory proteins that are known as inhibitors of NF-κB (IκBs). The IκB family of proteins consists of IκBα, IκBβ, IκBε and BCL-3 (Li, NRDD). An essential step in the activation of innate immune cells by pathogens, stress molecules and pro-inflammatory cytokines is the degradation of IκB and release of NF-κB and its subsequent phosphorylation allowing NF-κB proteins to translocate to the nucleus and bind to their cognate DNA binding sites to regulate the transcription of large numbers of genes. A crucial regulatory step in the degradation of IκBs is the signal-induced phosphorylation of IκB at specific amino-terminal serine residues, which is mediated by serine-specific IκB kinases (IKK). The serine phosphorylated IκB is then ubiquitinilated and degraded by the proteasome. The IKK complex consists of several proteins, the main ones being IKK1 (IKKγ), IKK2 and the regulatory subunit NF-κB essential modulator (NEMO, also known as IKKγ).

Inhibition of NF-κB activation can be accomplished by several strategies including, but not limited to, direct targeting the DNA-binding activity of individual NF-κB proteins using small molecules or decoy oligonucleotides; treatment with cell membrane-permeable non-degradable IκBα, -β or -ε mutant protein(s); blocking the nuclear translocation of NF-κB dimers by inhibiting the nuclear import system; stabilizing IκBα, -β or -ε protein(s) by developing ubiquitylation and proteasome inhibitors; targeting signaling kinases such as IKK using small-molecule inhibitors (Li et al., 2002); and treatment with cell membrane-permeable dominant negative IKK protein. Several drugs that are used to treat inflammatory diseases have effects on NF-κB activity such as glucocorticosteroids (GCS), aspirin and other anti-inflammatory drugs. Although these drugs do not target NF-κB specifically, parts of their pharmacologic effects are due to inhibition of NF-κB activity. Besides these drugs, many compounds have been described in literature as inhibitors of NF-κB activation, such as, for example, anti-oxidants, proteasome and protease inhibitors, IκB phosphorylation and/or degradation inhibitors and miscellaneous inhibitors (Table 1). It will be clear to a person skilled in the art that functional analogues to the compounds as listed in Table 1 can also be used to inhibit NF-κB activation. A functional analogue exhibits the same inhibitory activity of NF-κB activation in kind if not in amount.

Inhibition of MAPK/AP-1 Signal Transduction Pathway

Mammals express at least four distinctly regulated groups of mitogen-activated protein kinases (MAPKs), ERK-1/2, ERK5, JNK1/2/3 and p38α/β/γ/δ, that have been shown to regulate several physiological and pathological cellular phenomena, including inflammation, apoptotic cell death, oncogenic transformation, tumor cell invasion and metastasis (Herlaar et al., 1999). Upon cellular stimulation, a kinase cascade is initiated that ultimately leads to altered gene expression and consequently a biological response. The three main kinases in the MAPK cascades are the MAPK kinase kinase (MKKK), MAPK kinase (MKK) and MAPK. In total, 12 MAPK isoforms have been identified that can phosphorylate and activate a large range of substrates, including transcription factors and kinases. p38MAPK and JNK are stress-activated protein kinases that mediate responses to cellular stress factors such as UV light and oxidative stress. A wide variety of inflammatory mediators, such as cytokines, activate p38 MAPK in immune- and inflammatory cells. To date, several specific MAPK inhibitors have been developed in particular targeting p38 MAPK. The pyridinylimidazole compounds, exemplified by SB 203580, have been demonstrated to be selective inhibitors of p38 MAPK. This compound specifically inhibits p38α,β and β2 MAPK and has shown activity in a variety of animal models of acute and chronic inflammation. Other small molecule compounds that inhibit p38 MAPK are VX-745 (Vertex Pharmaceuticals), RWJ67657 (Johnson & Johnson) and HEP 689 (Leo Pharmaceuticals). Interestingly, SB 203580 has been shown to inhibit the maturation of dendritic cells. Other compounds that have been shown to inhibit dendritic cell maturation through inhibition of p38 MAPK are the anti-inflammatory sesquiterpene lactone parthenolide (PTL) and the cytokine IL-10. In contrast, inhibition of the ERK MAPK pathway by the selective inhibitors PD98059 and U0126, has been shown to enhance phenotypic and functional maturation of dendritic cells.

Little is known about the role of JNK MAPK in the regulation of innate- and adaptive immune responses. The JNK inhibitor SP600125 has been shown to inhibit the induction of IL-18 production by macrophages and the signaling of the T1/ST2, a cell membrane receptor that is selectively expressed on Th2 lymphocytes.

MAPKs are upstream regulators of AP-1. The transcription factor family activator protein 1 (AP-1) is formed by heterodimeric complexes of a Fos protein (c-Fos, Fra-1, Fra-2, FosB and FosB2) with a Jun protein (c-Jun, JunB and JunD) or a homodimer between two Jun proteins (Foletta et al., 1998). AP-1 regulates many of the genes up-regulated during immune- and inflammatory responses. The most well-known repressor of the transcription factor AP-1 are glucocorticoids. Together with the inhibition NF-κB activation by glucocorticoids, these are the major mechanisms for the anti-inflammatory effects of this drug class (McKay et al., 1999). Activation of gene transcription by AP-1 can also be inhibited by decoy oligonucleotides. It will be clear to a person skilled in the art that functional analogues to the compounds as mentioned above and used for the inhibition of the MAPK/AP-1 pathway can also be used to inhibit MAPK/AP-1 pathway activation. A functional analogue exhibits the same inhibitory activity of the MAPKIAP-1 pathway in kind, if not in amount.

Indirect Inhibition of NF-κB and MAPK/AP-1 Signal Transduction Pathways

The activation of the NF-κB and/or MAPK/AP-1 pathways can also be prevented by interference with “co-stimulation” molecules or locally produced activating mediators by (i) blocking of NF-κB and/or MAPK/AP-1-activating mediators, including, but not limited to, cytokines such as IL-1, -2, -12, -15, -17, -18, LIF, and members of the TNF super-family such as FAS ligand, GITR ligand, THANK, RANK ligand (also called TRANCE or OPGL), TNFα and TNFβ or blocking their specific cell membrane receptors, or (ii) blocking PRR including, but not limited to, toll-like receptors, lectin receptors or NODs, or (iii) prevention of oxidative stress using anti-oxidants, or (iv) blocking extra-cellular heat-shock proteins or their cell membrane receptors, or (v) blocking purinergic receptors, in particular, those expressed on APCs.

NF-κB activation, in particular in APCs, can also be inhibited by compounds that increase intracellular levels of cyclic AMP including, but not limited to, β2-adrenoceptor agonists, prostanoid EP2- or DP receptor agonists or phosphodiesterase IV inhibitors.

Inhibition of NF-κB and MAPK/AP−1 Signal Transduction Pathways by PPAR Activation

Recently, an interesting family of nuclear hormone receptors have emerged called peroxisome proliferator-activated receptors (PPARs) that, upon ligation, exert potent inhibitory effects on the transcription factors NF-κB and AP-1 (Daynes et al., 2002; Hihi et al., 2002). So far, three PPAR isoforms have been identified PPARα, PPARβ/δ and PPARγ with a high degree of sequence and structural homology. PPARs share the property of forming heterodimers with another nuclear receptor of the same subgroup, the 9-cis-retinoic acid receptor (RXR), which appears to be essential for their biological function. Various types of fatty acids and eicosanoids can bind to and activate PPARs, with some degree of isoform specificity (Daynes et al., 2002; Hihi et al., 2002). PPARα can be activated by α-linoleic-, γ-linoleic-, arachidonic- and eicosapentaenoic acids and by medium-chain saturated and monounsaturated fatty acids such as palmitic and oleic acids. PPARα can be activated selectively by LTB4 and 8(S)HETE. PPARγ is activated by α-linoleic-, γ-linoleic-, arachidonic- and eicosapentaenoic acids, although these endogenous ligands are weak activators. PPARγ is best stimulated by 9-HODE, 13-HODE and 15dPGJ2 and by the synthetic compound rosiglitazone and thiazolidinedione class of drugs. PPARβ/δ can be activated by some saturated, monounsaturated and unsaturated fatty acids, and by various eicosanoids including PGA1 and PGD2 and prostacyclin or a stable synthetic form. Interestingly, PPARα and PPARγ are expressed in antigen-presenting cells (monocytes, macrophages, dendritic cells and B-cells) and can play an important role in down-regulation of NF-κB and AP-1 activity (Daynes et al., 2002; Nencioni et al., 2002).

DNA Vaccination

The standard DNA vaccine consists of the specific gene(s) of interest cloned into a bacterial plasmid engineered for optimal expression in eukaryotic cells. Essential features include a strong promoter for optimal expression in mammalian cells, an origin of replication allowing growth in bacteria, a bacterial antibiotic-resistance gene and incorporation of polyadenylation sequences to stabilize mRNA transcripts. Moreover, DNA vaccines also contain specific nucleotide sequences that play a critical role in the immunogenicity of these vaccines. In case of allergen vaccination using DNA vaccines, the plasmid contains nucleotide sequences encoding one or more allergens or allergen fragments containing at least one T-cell epitope sequence. Allergens for use in the invention include, but are not limited to, the list available on the World-Wide Web at http://www.allergen.org/List.htm. It has been shown that immune responses induced by DNA vaccination are mediated by APCs, in particular, DCs migrating from the site of vaccination to the draining lymph nodes. The DCs are either directly transfected or take up secreted protein from other transfected cells, i.e., myocytes. Fusion of the allergen or allergen fragment to an IgG Fc fragment improves the secretion of the encoding allergen and the subsequent targeting to and uptake by APCs. Targeting of DNA vaccines to APCs, in particular DCs, may be obtained by using particular viral vectors including, but not limited to, herpes virus, vaccinia virus, adenovirus, influenza virus, retroviruses and lentiviruses (Jenne et al., 2001). Second generation DNA vaccines are also being developed that introduce not only a gene encoding the target antigen, but also a gene encoding some other factor capable of inducing an altered immune response. Within the present invention, the plasmid comprising a T-cell epitope can be combined with genes encoding proteins that inhibit the activation of the NF-κB pathway, including, but not limited to, (non-degradable) IκB proteins or dominant negative forms of IKK proteins or NF-κB proteins and/or genes encoding proteins that inhibit the activation of the MAPK/AP-1 pathway, including, but not limited to, dominant negative forms of critical proteins leading to the activation of Fos and/or Jun proteins such as p38 MAPK or dominant negative forms of Fos and/or Jun proteins. In another embodiment, the plasmid comprising a T-cell epitope can be combined with small interfering RNA sequences or anti-sense sequences that inhibit the expression of IKK, NF-κB, p38 MAPK or AP-1 proteins.

Of course, the effects of inhibiting or preventing the production of a co-stimulator molecule in an antigen-presenting cell, when the antigen-presenting cell is contacted with an allergen, can also be studied and assessed on isolated cells in vitro. The effects of a compound on the production of a co-stimulator molecule can thus be tested in vitro and a selection can be made as to what compound is most suitable for inhibiting the production of a co-stimulator molecule by an antigen-presenting cells. Therefore, the present invention also teaches a method to inhibit and/or prevent the production of a co-stimulator molecule in an antigen-presenting cell in the presence of an allergen, wherein the production of a co-stimulator molecule is inhibited and/or prevented by inhibiting the NF-κB and/or the MAPK/AP-1 signal-transducing pathways in the antigen-presenting cell.

Novel Allergen Vaccination Strategies

In a recent WHO position paper, allergen vaccination or immunotherapy is defined as the practice of administering gradually increasing quantities of an allergen extract to an allergic subject to ameliorate the symptoms associated with subsequent exposure to the causative allergen (Bousquet et al., 1998). In the present invention, we describe novel forms of allergen vaccination that offer significant advantages over current allergen immunotherapy practice.

Allergens for use in the invention include, but are not limited to, the list available on the World-Wide Web at http://www.allergen.org/List.htm. The allergen used can be an allergen extract such as house-dust mite or pollen or fragments thereof containing at least one T-cell epitope or an entire or partial recombinant allergen protein such as Der p1 containing at least one cell epitope. The preferred route of administration is subcutaneous injection, however, other routes, such as nasal, oral or sublingual application, can be effective as well. Another embodiment is the use of DNA vaccines that incorporate a gene encoding the entire or partial allergen sequence and containing at least one T-cell epitope sequence (Walker et al., 2001).

An allergen vaccination course usually involves a build-up phase (increasing allergen dose) and a maintenance phase (maximum dosage of the allergen) in which the allergen is administered with a 1 to 2 month interval. The duration of allergen vaccination required to maintain improvement in clinical symptoms has been advised to 3 to 5 years of therapy (Bousquet et at., 1998). Allergen vaccination is rarely started before the age of 5 years. When started early in the disease process, allergen vaccination may modify the progression of the disease.

Novel allergen vaccination strategies consist of the treatment with one or more compounds that inhibit the NF-κB pathway and/or the MAPK/AP-1 pathway at the time of allergen injection. This/these compound(s) may be co-injected subcutaneously together with the allergen or given separately by systemic, enteral, or parenteral administration. A non-exhaustive list of inhibitors of NF-κB activation is provided in Table 1. In case of DNA vaccination, the plasmid comprising a T-cell epitope can be combined with genes that inhibit the NF-κB and/or the MAPK/AP-1 pathway.

The methods provided herein are suitable for treating any allergic disorders including, but not limited to, rhinitis, food allergy, urticaria, atopic dermatitis and asthma.

TABLE 1
Non exhaustive list of inhibitors of NF-κB activation as described in
literature, grouped as anti-oxidants, protease and protease inhibitors,
IKβA phosphorylation and/or degradation inhibitors and miscellaneous
inhibitors (modified from http://people.bu.edu/gilmore/nf-kb).

| | | IκBα phosphorylation | |
|---|---|---|---|
| | proteasome and/or | and/or degradation | miscellaneous |
| anti-oxidants | protease inhibitors | inhibitors | inhibitors |
| α-lipoic acid | ALLnL | Rocaglamides (Aglaia | β-amyloid protein |
| | (N-acetyl-leucinyl- | derivatives) | |
| | leucinyl-norleucinal, | | |
| | MG101) | | |
| α-tocopherol | Z-LLnV | Jesterone dimer | Glucocorticoids |
| | (carbobenzoxyl- | | |
| | leucinyl-leucinyl- | | |
| | norvalinal, MG115) | | |
| Aged garlic extract | Z-LLL | Silibinin | IL-10 |
| | (carbobenzoxyl- | | |
| | leucinyl-leucinyl- | | |
| | leucinal, MG132) | | |
| Anetholdithiolthione | Lactacystine, | Quercetin | IL-13 |
| (ADT) | β-lactone | | |
| Butylated | Boronic Acid Peptide | Staurosporine | IL-11 |
| hydroxyanisole | | | |
| (BHA) | | | |
| Cepharanthine | Ubiquitin Ligase | Aspirin, sodium salicylate | Dioxin |
| | Inhibitors | | |
| Caffeic Acid | PS-341 | BAY-117821 | Leptomycin B |
| Phenethyl Ester | | (E3((4-methylphenyl)- | (LMB) |
| (3,4- | | sulfonyl)-2-propenenitrile) | |
| dihydroxycinnamic | | | |
| acid, CAPE) | | | |
| Catechol Derivatives | Cyclosporin A | BAY-117083 | NLS Cell permeable |
| | | (E3((4-t-butylphenyl)- | peptides |
| | | sulfonyl)-2-propenenitrile) | |
| Dibenzylbutyrol- | FK506 | Cycloepoxydon; | o,o′-bismyristoyl |
| actone lignans | (Tacrolimus) | 1-hydroxy-2- | thiamine disulfide |
| | | hydroxymethyl-3-pent-1- | (BMT) |
| | | enylbenzene | |
| Diethyldithio- | Deoxyspergualin | Extensively oxidized low | ADP ribosylation |
| carbamate (DDC) | | density lipoprotein | inhibitors |
| | | (ox-LDL), | (nicotinamide, 3- |
| | | 4-Hydroxynonenal (HNE) | aminobenzamide) |
| Diferoxamine | APNE (N-acetyl-DL- | Ibuprofen | Atrial Natriuretic |
| | phenylalanine- | | Peptide (ANP) |
| | b-naphthylester) | | |
| Dihydrolipoic Acid | BTEE (N-benzoyl | Nitric Oxide (NO) | Atrovastat |
| | L-tyrosine-ethylester) | | (HMG-CoA |
| | | | reductase inhibitor) |
| Disulfiram | DCIC (3,4- | Prostaglandin A1 | AvrA protein |
| | dichloroisocoumarin) | | (Salmonella) |
| Dimethyldithio- | DFP (diisopropyl | Sulfasalazine | Bovine serum |
| carbamates | fluorophosphate) | | albumin |
| (DMDTC) | | | |
| Curcumin | TPCK (N-a-tosyl-L- | YopJ (encoded by Yersinia | Calcitriol (1α,25- |
| (Diferulolylmethane) | phenylalanine | pseudotuberculosis) | dihydroxyvitamine |
| | chloromethyl ketone) | | D3) or analogs |
| Ebselen | TLCK | A-melanocyte-stimulating | Capsiate |
| | (N-a-tosyl-L-lysine | hormone (α-MSH) | |
| | chloromethyl ketone) | | |
| EPC-K1 | Phosphodiesterase | Aucubin | Catalposide |
| (phosphodiester | inhibitors, i.e., | | |
| compound of | theophylline; | | |
| vitamin E and | pentoxyphylline | | |
| vitamin C) | | | |
| Epigallocatechin-3- | | β-lapachone | Clarithromycin |
| gallate (EGCG; | | | |
| green tea | | | |
| polyphenols) | | | |
| Ethylene Glycol | | Capsaicin (8- | Diamide |
| Tetraacetic Acid | | methyl-N-vanillyl-6- | |
| (EGTA) | | nonenamide) | |
| Gamma- | | Core Protein of | E3330 (quinone |
| glutamylcysteine | | Hepatitis C virus | derivative) |
| synthetase | | (HCV) | |
| (gamma-GCS) | | | |
| Glutathione | | Diamide (tyrosine | Epoxyquinol A |
| | | phosphatase inhibitor) | (fungal metabolite) |
| IRFI 042 | | E-73 | Glycyrrhizin |
| | | (cycloheximide analog) | |
| Iron tetrakis | | Emodin (3-methyl- | Hematein (plant |
| | | 1,6,8- | compound) |
| | | trihydroxyanthraquinone) | |
| L-cysteine | | Erbstatin (tyrosine kinase | Herbimycin A |
| | | inhibitor) | |
| Lacidipine | | Estrogen (E2) | Hypericin |
| Magnolol | | Fungal gliotoxin | Hydroquinone |
| | | | (HQ) |
| Manganese | | Genistein (tyrosine kinase | IL-4 |
| Superoxide | | inhibitor) | |
| Dismutase | | | |
| (Mn-SOD) | | | |
| Melatonin | | IL-13 | 1κB-like proteins |
| | | | (encoded by ASFV) |
| N-acetyl-L-cysteine | | Leflunomide metabolite | Kamebakaurin |
| (NAC) | | (A77 1726) | |
| Nordihydro- | | Neurofibromatosis- | KT-90 (morphine |
| guaiaritic acid | | 2 (NF-2) protein | synthetic derivative) |
| (NDGA) | | | |
| Ortho- | | Pervanadate (tyrosine | Metals (chromium, |
| phenanthroline | | phosphatase inhibitor) | cadmium, gold, lead, |
| | | | mercury, zinc, |
| | | | arsenic) |
| Phenylarsine oxide | | Phenylarsine oxide (PAO, | Mevinolin, 5′- |
| (PAO, tyrosine | | tyrosine phosphatase | methylthioadenosine |
| phosphatase | | inhibitor) | (MTA) |
| inhibitor) | | | |
| Pyrrolinedithio- | | Pituitary adenylate | N-ethyl-maleimide |
| carbamate (PDTC) | | cyclase-activating | (NEM) |
| | | polypeptide | |
| | | (PACAP) | |
| Quercetin | | Resiniferatoxin | Nicotine |
| Red wine | | Sesquiterpene lactones | 1,2,3,4,6-penta-O- |
| | | (parthenolide) | galloyl-beta-D- |
| | | | glucose |
| Rotenone | | Thiopental | Pentoxifylline |
| | | | (1-(5′-oxohexyl) |
| | | | 3,7-dimetylxanthine, |
| | | | PTX) |
| S-allyl-cysteine | | Triglyceride-rich | Phenyl-N-tert- |
| (SAC, garlic | | lipoproteins | butylnitrone |
| compound) | | | (PBN) |
| Tepoxaline | | Vasoactive | Pyrithione |
| (5-(4-chlorophenyl)- | | intestinal peptide | |
| N-hydroxy-(4- | | | |
| methoxyphenyl)- | | | |
| N-methyl-1H- | | | |
| pyrazole-3- | | | |
| propanamide) | | | |
| Vitamin C | | HIV-1 Vpu protein | Rolipram |
| Vitamin E | | Dibenzyl butyrolactone | Quinadril (ACE |
| derivatives | | lignans | inhibitor) |
| a-torphryl succinate | | Aurintricarboxylic acid | Ribavirin |
| a-torphryl acetate | | BAY 11-7082 | Secretory leukocyte |
| | | | protease inhibitor |
| | | | (SLPI) |
| PMC (2,2,5,7,8- | | BAY 11-7085 | Serotonin derivative |
| pentamethyl-6- | | | (N-(p-coumaroyl) |
| hydroxychromane) | | | serotonin, SC) |
| Carnosol | | IKK-NBD peptide | Silymarin |
| Sodium selenite | | Piceatannol | Sulfasalazine |
| Mol 294 | | | Vascular endothelial |
| | | | growth factor |
| | | | (VEGF) |
| | | | D609 |
| | | | (phosphatidylcholine- |
| | | | phospholipase |
| | | | C inhibitor) |
| | | | ethyl 2-[(3-methyl- |
| | | | 2,5-dioxo(3- |
| | | | pyrrolinyl))amino]- |
| | | | 4-(trifluoromethyl) |
| | | | pyrimidine-5- |
| | | | carboxylate |
| | | | Cycloprodigiosin |
| | | | hydrochloride |
| | | | RO31-8220 (PKC |
| | | | inhibitor) |
| | | | SB203580 (p38 |
| | | | MAPK inhibitor) |
| | | | Tranilast [N-(3,4- |
| | | | dimethoxycinnamoyl) |
| | | | anthranilic acid] |
| | | | Triptolide (PG490, |
| | | | extract of Chinese |
| | | | herb) |
| | | | LY294,002 |
| | | | Mesalamine |
| | | | Qingkailing and |
| | | | Shuanghuanglian |
| | | | (Chinese medicinal |
| | | | preparations) |
| | | | Tetrathio-molybdate |
| | | | Na⁺/H⁺ exchange |
| | | | inhibitors i.e., |
| | | | amiloride |
| | | | Gliotoxin |
| | | | Estriol |
| | | | Wortmannin (fungal |
| | | | metabolite) |
| | | | Inducers of heat- |
| | | | shock proteins, i.e., |
| | | | curcumin |
| | | | Helenalin |

The invention is further explained in more detail in the following description, which is not limiting the invention.

### Experimental Part

### Materials and Methods

Animals. Animal care and use were performed in accordance with the guidelines of the Dutch Committee of Animal Experiments. Specific pathogen-free male BALB/c mice (5 to 6 weeks old) were purchased from Charles River (Maastricht, The Netherlands) and housed in macrolon cages in a laminar flow cabinet and provided with food and water ad libitum.

Sensitization, treatment and challenge. Mice (6 to 8 weeks old) were sensitized intraperitoneally (i.p.) on days 0 and 7 with 10 μg ovalbumin (OVA, grade V, Sigma-Aldrich) in 0.1 ml alum (Pierce, Rockford, Ill.). Two weeks after the last sensitization, the mice were divided into six groups. The sham-immunotherapy and the OVA-immunotherapy groups were treated with three s.c. injections of, respectively, 0.2 ml pyrogen-free saline (B. Braun, Melsungen, Germany) or 1 mg OVA in 0.2 ml pyrogen-free saline on alternate days. In three groups, OVA-immunotherapy was co-injected with 0.1 μg, 0.03 μg or 0.01 μg 1α,25-dihydroxyvitamin D3 (1α,25(OH)2 VitD3), a selective inhibitor of NF-κb. One group was treated with sham-immunotherapy and combined with co-injection of 0.1 μg 1α,25(OH)2 VitD3. Ten days after treatment, mice were exposed to three OVA inhalation challenges (10 mg/ml in saline) for 20 minutes every third day.

In a second series of experiments, OVA-immunotherapy, as described above, was carried out using a sub-optimal amount of 100 μg OVA in saline for OVA-immunotherapy. OVA-immunotherapy was either given alone or in combination with 0.01 μg 1α,25(OH)2 VitD3. Sham-immunotherapy alone or in combination with 0.01 μg 1α,25(OH)2 VitD3 served as control groups.

Measurement of airway responsiveness in vivo. Airway responsiveness to methacholine was measured after treatment but before OVA challenge (pre-measurement) and 24 hours after the last OVA challenge. Airway responsiveness was measured in conscious, unrestrained mice using barometric whole-body plethysmography by recording respiratory pressure curves (Buxco, EMKA Technologies, Paris, France) in response to inhaled methacholine (acetyl-β-methylcholine chloride, Sigma-Aldrich). Airway responsiveness was expressed in enhanced pause (Penh), as described in detail previously (Deurloo et al., 2001). Briefly, mice were placed in a whole-body chamber and basal readings were determined for three minutes. Aerosolized saline, followed by doubling concentrations of methacholine (ranging from 3.13-25 mg/ml in saline), were nebulized for three minutes, and readings were determined for three minutes after each nebulization.

Determination of OVA-specific IgE levels in serum. From each mouse, serum was obtained after treatment but before OVA challenge (pre-measurement) by a small incision in the tail vein. After measurement of airway responsiveness in vivo, mice were sacrificed by i.p. injection of 1 ml 10% urethane in saline and were bled by cardiac puncture. Subsequently, serum was collected and stored at −70° C. until analysis. Serum levels of OVA-specific IgE were measured by sandwich ELISA as described previously (Deurloo et al., 2001).

Analysis of the cellular composition of the bronchoalveolar lavage fluid. Bronchoalveolar lavage (BAL) was performed immediately after bleeding of the mice by lavage of the airways through a tracheal cannule five times with 1 ml saline (37° C.). Cells in the BALF were centrifuged and resuspended in cold PBS. The total number of cells in the BAL was determined using a Bürker-Türk counting chamber (Karl Hecht Assistant KG, Sondheim/Röhm, Germany). For differential BAL cell counts, cytospin preparations were made (15×g, five minutes, 4° C., Kendro Heraues Instruments, Asheville, N.C.). Next, cells were fixed and stained with Diff-Quick (Dade A. G., Düdingen, Switzerland). Per cytospin, 200 cells were counted and differentiated into mononuclear cells, eosinophils, and neutrophils by standard morphology and staining characteristics.

Statistical analysis. All data are expressed as mean±standard error of mean (SEM). The airway dose-response curves to methacholine were statistically analyzed by a general linear model of repeated measurements followed by post-hoc comparison between groups. Data were log transformed before analysis to equalize variances in all groups. All other data were analyzed using a Student's t test (2-tailed, homosedastic). Results were considered statistically significant at the p<0.05 level.

### Results 1

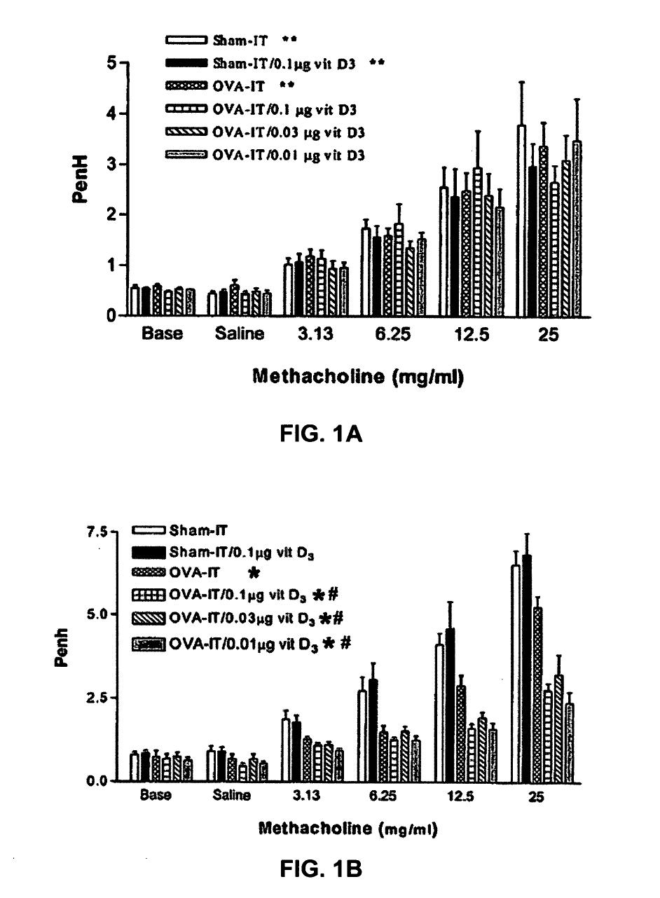

Airway responsiveness in vivo. No significant differences between all six groups were observed in airway responsiveness to methacholine after treatment but prior to OVA challenge (FIG. 1A). OVA-sensitized BALB/c mice that received sham-immunotherapy displayed significant airway hyper-responsiveness (AHR) to methacholine after OVA inhalation challenge as compared to before challenge. Mice that received OVA-immunotherapy displayed significant AHR to methacholine after OVA challenge as compared to before challenge. However, OVA-immunotherapy partially reduced (P<0.05) AHR to methacholine as compared to sham-treated mice. Interestingly, co-injection of 1α,25(OH)2 VitD3, at all doses used, with OVA-immunotherapy significantly potentiated the reduction of AHR to methacholine compared to OVA-IT alone. Co-injection of 1α,25(OH)2 VitD3 with sham-immunotherapy did not affect AHR. It can be concluded that co-injection of the selective NF-κb inhibitor 1α,25(OH)2 VitD3 potentiates the suppressive effect of OVA-immunotherapy on AHR.

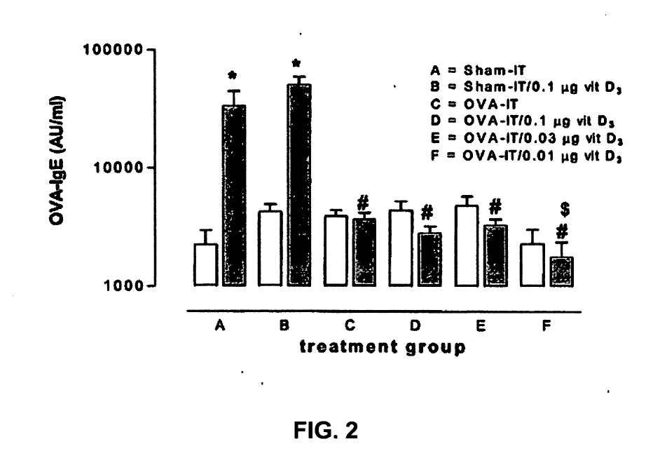

OVA-specific IgE levels in serum. OVA-sensitized BALB/c mice that received sham-immunotherapy showed a significant increase in serum levels of OVA-specific IgE after OVA inhalation challenge as compared to before challenge (FIG. 2). In mice that received OVA-immunotherapy, serum OVA-specific IgE levels were significantly reduced after OVA challenge as compared to sham-treated OVA-challenged mice. Co-injection of 0.01 μg 1α,25(OH)2 VitD3 with OVA-immunotherapy significantly increased the reduction of serum IgE levels as compared to OVA-immunotherapy alone. Co-injection of 1α,25(OH)2 VitD3 with sham-immunotherapy did not affect serum IgE levels as compared to sham-immunotherapy alone. It can be concluded that co-injection of the selective NF-κb inhibitor 1α,25(OH)2 VitD3, at 0.01 μg, potentiates the suppression of serum OVA-specific IgE levels after OVA-immunotherapy.

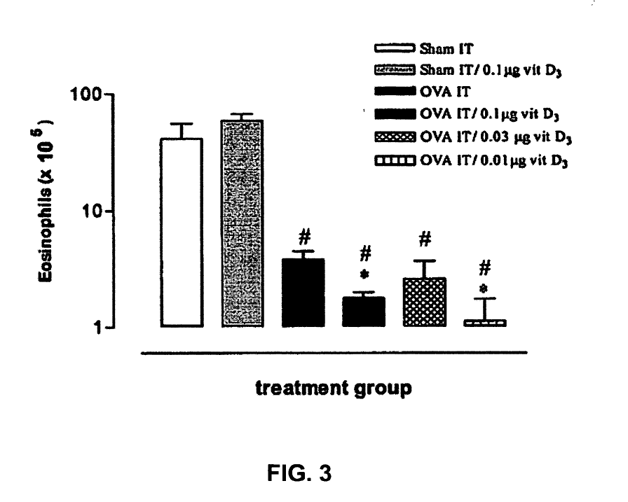

Cellular composition of the bronchoalveolar lavage fluid. In BALB/c mice, no eosinophils are present in BALF prior to OVA inhalation. OVA-sensitized BALB/c mice that received sham-immunotherapy showed BALF eosinophilia after OVA inhalation challenge (FIG. 3). After OVA challenge of mice that received OVA-immunotherapy, the number of BALF eosinophils were significantly reduced as compared to sham-treated mice. Co-injection of 0.01 μg 1α,25(OH)2 VitD3 with OVA-immunotherapy significantly increased the reduction of BALF eosinophil numbers as compared to OVA-immunotherapy alone. Co-injection of 1α,25(OH)2 VitD3 with sham-immunotherapy did not significantly affect BAL eosinophil number. It can be concluded that co-injection of the selective NF-κb inhibitor 1α,25(OH)2 VitD3, at 0.01 μg, potentiates the suppression of BALF eosinophilia after OVA-immunotherapy.

Collectively, it is concluded that the selective NF-κb inhibitor 1α,25(OH)2 VitD3 is able to potentiate the beneficial effect of OVA-immunotherapy on airway hyper-responsiveness, serum IgE levels and airway eosinophilia, all important hallmarks of human asthma.

### Results 2

In the results described herein, the effect of co-injection of the selective NF-κB inhibitor 1α,25(OH)2 VitD3 with OVA-immunotherapy was most pronounced with regards to potentiation of the suppression of AHR. The potentiation of the suppression of BALF eosinophilia was less pronounced because the amount of OVA used for OVA-immunotherapy already induced a strong, almost maximal, suppression of BALF eosinophil numbers (FIG. 3). Therefore, we examined the effect of co-injection of 0.01 μg 1α,25(OH)2 VitD3 with a sub-optimal amount of OVA (100 μg) for OVA-immunotherapy.

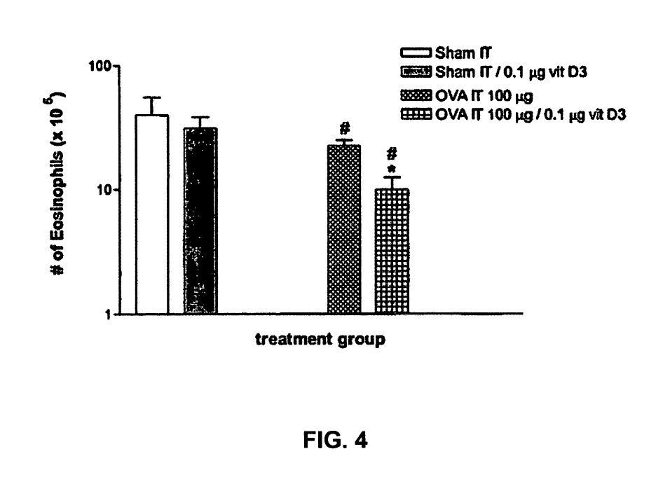

Cellular composition of the bronchoalveolar lavage fluid. In BALB/c mice, no eosinophils are present in BALF prior to OVA inhalation. OVA-sensitized BALB/c mice that received sham-immunotherapy showed BALF eosinophilia after OVA inhalation challenge (FIG. 4). After OVA challenge of mice that received OVA-immunotherapy (100 μg), the number of BALF eosinophils were significantly reduced as compared to sham-treated mice. Co-injection of 0.01 μg 1α,25(OH)2 VitD3 with a sub-optimal dose of OVA-immunotherapy significantly increased the reduction of BALF eosinophil numbers as compared to OVA-immunotherapy alone. Co-injection of 1α,25(OH)2 VitD3 with sham-immunotherapy did not significantly affect BAL eosinophil number.

It can be concluded that co-injection of the selective NF-κb inhibitor 1α,25(OH)2 VitD3 with sub-optimal OVA-immunotherapy is able to potentiate the suppression of BAL eosinophil numbers by a sub-optimal dose of OVA. Furthermore, co-injection of the selective NF-κb inhibitor 1α,25(OH)2 VitD3 can reduce the amount of allergen (in this case OVA) needed to obtain a particular level of suppression by allergen immunotherapy.

### REFERENCES

Baxter A. G. and P. D. Hodgkin (2002). Activation rules: the two-signal theories of immune activation. Nat. Rev. Immunol. 2, 439-46.
Bousquet J., R. Lockey, H. J. Mailing, E. Alvarez-Cuesta, G. W. Canonica, M. D. Chapman, P. J. Creticos, J. M. Dayer, S. R. Durham, P. Demoly, R. J. Goldstein, T. Ishikawa, K. Ito, D. Kraft, P. H. Lambert, H. Lowenstein, U. Muller, P. S. Norman, R. E. Reisman, R. Valenta, E. Valovirta and H. Yssel (1998). Allergen immunotherapy: therapeutic vaccines for allergic diseases. World Health Organization. American Academy of Allergy, Asthma and Immunology. Ann. Allergy Asthma Immunol. 81, 401-5.
Daynes R. A. and D. C. Jones (2002). Emerging roles of ppars in inflammation and immunity. Nat. Rev. Immunol. 2, 748-59.
Dhodapkar M. V., R. M. Steinman, J. Krasovsky, C. Munz and N. Bhardwaj (2001). Antigen-specific Inhibition of Effector T-Cell Function in Humans after Injection of Immature Dendritic Cells. J. Exp. Med. 193, 233-238.
Foletta V. C., D. H. Segal and D. R. Cohen (1998). Transcriptional regulation in the immune system: all roads lead to AP-1. J. Leukoc. Biol. 63, 139-52.
Gallucci S. and P. Matzinger (2001). Danger signals: SOS to the immune system. Curr. Opin. Immunol. 13, 114-119.
Herlaar E. and Z. Brown (1999). p38 MAPK signaling cascades in inflammatory disease. Mol. Med. Today 5, 439-47.
Hihi A. K., L. Michalik and W. Wahli (2002). PPARs: transcriptional effectors of fatty acids and their derivatives. Cell. Mol. Life Sci. 59, 790-8.
Jenne L., G. Schuler and A. Steinkasserer (2001). Viral vectors for dendritic cell-based immunotherapy. Trends Immunol. 22, 102-107.
Jonuleit H., E. Schmitt, K. Steinbrink and A. H. Enk (2001). Dendritic cells as a tool to induce anergic and regulatory T-cells. Trends Immunol. 22, 394-400.
Li Q. and I. M. Verma (2002). NF-κB regulation in the immune system. Nat. Rev. Immunol. 2, 725-34.
Matzinger P. (2002). The danger model: a renewed sense of self Science 296, 301-5.
McKay L. I. and J. A. Cidlowski (1999). Molecular control of immune/inflammatory responses: interactions between nuclear factor-kappa B and steroid receptor-signaling pathways. Endocr. Rev. 20, 435-59.
Nencioni A., F. Grunebach, A. Zobywlaski, C. Denzlinger, W. Brugger and P. Brossart (2002). Dendritic cell immunogenicity is regulated by peroxisome proliferator-activated receptor gamma. J. Immunol. 169, 1228-35.
Pulendran B., K. Palucka and J. Banchereau (2001). Sensing pathogens and tuning immune responses. Science 293, 253-6.
Reis e Sousa C. (2001). Dendritic cells as sensors of infection. Immunity 14, 495-8.
Roncarolo M. G., M. K. Levings and C. Traversari (2001). Differentiation of T Regulatory Cells by Immature Dendritic Cells. J. Exp. Med. 193, F5-F10.
Walker C. and C. Zuany-Amorim (2001). New trends in immunotherapy to prevent atopic diseases. Trends Pharmacol. Sci. 22, 84-90.

1. A method to inducing and/or increasing tolerance to an allergen in a subject, said method comprising:
inhibiting and/or preventing production of a co-stimulator molecule in an antigen-presenting cell in an allergen's presence.

2. The method according to claim 1, wherein said production of a co-stimulator molecule is inhibited and/or prevented by inhibiting NF-κB and/or MAPK/AP-1 signal-transducing pathways in the antigen-presenting cell.

3. The method according to claim 2, comprising:
inhibiting transcription of genes involved in activation of the NF-κB and/or the MAPK/AP-1 signal-transducing pathways in an antigen-presenting cell.

4. The method according to claim 2, wherein the NF-κB and/or the MAPK/AP-1 signal-transducing pathways are inhibited by a ligand to a peroxisome proliferator-activated receptor.

5. The method according to claim 2, wherein the NF-κB-transducing pathway is inhibited by at least one antioxidant compound, proteasome, protease inhibitor, IκB phosphorylation, degradation inhibitor, or mixture thereof.

6. The method according to claim 2, wherein said NF-κB-transducing pathway is inhibited by at least one compound selected from the group of a non-steroidal anti-inflammatory compound, a glucocorticosteroid compound, a di-hydroxyvitamin D3 compound, a cAMP-elevating compound, and mixtures thereof.

7. The method according to claim 2, wherein the MAPK/AP-1 signal-transducing pathway is inhibited by a compound selected from the group consisting of a non-steroidal anti-inflammatory compound, a steroidal anti-inflammatory compound, a pyridinylimidazole compound, a NF-κB decoy oligonucleotide, an AP-1 decoy oligonucleotide, and mixtures thereof.

8. A pharmaceutical composition comprising:
an inhibitor of NF-κB and/or MAPK/AP-1 signal-transducing pathway,
one or more allergens, and
a diluent suitable for pharmaceutical administration.

9. The pharmaceutical composition of claim 8, wherein said inhibitor of the NF-κB and/or the MAPK/AP-1 signal-transducing pathway is combined with said one or more allergens before administration to a subject.

10. A method of increasing induction of immunotolerance in a subject, said method comprising:
administering the pharmaceutical composition of claim 8 by oral, enteral, intranasal, and/or dermal administration to the subject,
thus inducing immunotolerance in the subject.

11. A method of increasing induction of immunotolerance in a subject, said method comprising:
providing, to the subject, an inhibitor of NF-κB and/or MAPK/AP-1 signal-transducing pathway by oral, enteral, intranasal, and/or dermal administration, and
administering an allergen to the subject.

12. A vaccine comprising:
a nucleic acid sequence encoding one or more allergen sequences.

13. A method for treating an allergic disease in a subject in perceived need thereof, the method comprising:
administering to the subject the vaccine of claim 12; and
inhibiting production of a co-stimulator molecule in an antigen-presenting cell.

14. The vaccine of claim 12, further comprising:
at least one nucleotide sequence encoding a protein that inhibits activation of NF-κB and/or MAPK/AP-1 signal-transducing pathway.

15. The vaccine of claim 12, further comprising:
at least one small interfering RNA sequence and/or antisense sequence that inhibits expression of NF-κB protein, AP-1 protein, or both NF-κB and AP-1 proteins.

16. A method of treating or preventing allergic disease in a subject in perceived need thereof, the method comprising:
administering to the subject the vaccine of claim 14.

17. A method of treating or preventing allergic disease in a subject in perceived need thereof, the method comprising:
administering to the subject the vaccine of claim 15.

18. A method of inhibiting and/or preventing production of a co-stimulator molecule in an antigen-presenting cell in the presence of an allergen, said method comprising:
inhibiting NF-κB and/or MAPK/AP-1 signal-transducing pathways in the antigen-presenting cell.

19. A method of increasing induction of immunotolerance in a subject, said method comprising:
administering to the subject the pharmaceutical composition of claim 9 by oral, enteral, intranasal, and/or dermal administration, thus inducing immunotolerance in the subject.

20. The method according to claim 4, wherein the MAPK/AP-1 signal-transducing pathway is inhibited by a compound selected from the group consisting of a non-steroidal anti-inflammatory compound, a steroidal anti-inflammatory compound, a pyridinylimidazole compound, an NF-κB decoy oligonucleotide, an AP-1 decoy oligonucleotide, and mixtures thereof.
